(19) United States
(12) Patent Application Publication
(10) Pub. No.: US 2025/0243605 A1
(43) Pub. Date: Jul. 31, 2025

(54) METHOD FOR MANUFACTURING A PHOTOCATALYTIC MULTI-COMPONENT FIBER AND PHOTOCATALYTIC MULTI-COMPONENT FIBER

(71) Applicant: PureNat, Anglet (FR)

(72) Inventor: Natacha KINADJIAN CAPLAT, Anglet (FR)

(21) Appl. No.: 18/845,917

(22) PCT Filed: Mar. 10, 2023
(86) PCT No.: PCT/EP2023/056191

§ 371 Date: Sep. 11, 2024

(30) Foreign Application Priority Data
Mar. 11, 2022 (FR) FR2202158

(51) Int. Cl. D01F 1/10; B01D 39/16; B01D 46/00; B01D 53/00; B01D 53/88; B01J 21/06; B01J 31/06; B01J 31/38; B01J 35/39; B01J 35/40; B01J 35/58; B01J 37/00; B01J 37/08; D01D 5/32; D01D 5/34; D01D 5/36; D01D 10/02; D01F 6/06

(52) U.S. Cl. CPC D01F 1/10; B01D 39/1623; B01D 46/0027; B01D 53/007; B01D 53/885; B01J 21/063; B01J 31/06; B01J 31/38; B01J 35/39; B01J 35/40; B01J 35/58; B01J 37/0009; B01J 37/082; D01D 5/32; D01D 5/34; D01D 5/36; D01D 10/02; D01F 6/06; B01D 2239/0225; B01D 2239/0233; B01D 2239/0407; B01D 2239/0618; B01D 2239/10; B01D 2255/20707; B01D 2255/802; B01D 2255/915; B01D 2258/06; B01D 2259/804; B01D 2279/35; D10B 2321/022; D10B 2401/00; D10B 2505/04

(57) ABSTRACT

The invention relates to a method (100) for manufacturing a photocatalytic multi-component fiber including the following steps:
Supplying a support mixture (110), including at least one thermoplastic polymer;
Supplying an active mixture (120), including:
   at least one organic polymer,
   at least one photocatalyst at a concentration of at least 10% by weight relative to the weight of active mixture,
   at least one coupling agent resistant to oxidation,
Spinning (130) a multi-component fiber from support and active mixtures;
Eliminating (160) the at least one organic polymer on the surface of the multi-component fiber so as to generate a photocatalytic multi-component fiber.

The invention also relates to a multi-component fiber, a textile and a filter.

### TECHNICAL FIELD

The invention relates to the field of textiles. In particular, the invention relates to the field of textile fibers.

The invention relates more particularly to a method for manufacturing a photocatalytic multi-component fiber as well as a photocatalytic multi-component fiber as such. Furthermore, the invention relates to a textile including a photocatalytic multi-component fiber as well as a filter provided with at least one textile according to the invention.

### PRIOR ART

The known prior art from which the invention was developed is described below.

Air and water pollution have continued to increase in recent years. The presence of organic or inorganic molecules in the air and/or water is carefully monitored. Indeed, it has been proven that air pollution causes, on the one hand, numerous pathologies (cardiorespiratory or else certain cancers for example) but also aggravated deterioration and pollution of the environment. Likewise, water pollution (water table, lake, river, sea and ocean) continues to increase to the detriment of fauna and flora. Also, numerous purification apparatuses (air and/or water) have been developed. These devices are intended in particular for the elimination of pollutants, pathogens or else allergens.

These purification apparatuses generally operate by a physical method, for example by filter or electrostatic dust collector, or by absorption of pollutants on materials (such as activated carbons for example), or by destruction or inactivation using ultraviolet rays or by photocatalysis. Some systems combine more than one technology and each purification device may have different benefits and risks.

In the field of air treatment, photocatalysts are materials that can decompose matter without emitting secondary pollutants. TiO₂ is one of the most used photocatalysts. It is a semiconductor material which allows various redox reactions including in particular the decomposition of pollutants using the characteristics related to the absorption of light and more precisely in the UV field. In addition, TiO₂, in addition to its strong photoactivity, is a stable material which has high yields and which, particularly advantageously for industry, is inexpensive.

The treatment method implemented by photocatalytic purifiers allows the decomposition and degradation of pollutants under the action of light rays on the surface of a photocatalyst, generally titanium dioxide (TiO₂). The method destroys volatile organic compounds, inorganic pollutants and microorganisms. The finalized process essentially produces water and carbon dioxide.

For photocatalytic purifiers, the photocatalyst is generally deposited on the surface of a substrate. The photocatalyst can be deposited in the form of powder, suspension or solution. This type of deposition on the surface of a substrate can lead to the formation of photocatalyst aggregates, and only partial coverage over small thicknesses. In this context, the photocatalyst tends to detach from its substrate easily, which makes the substrate on which it was deposited very quickly inactive.

Furthermore, binders that can be added to improve the adhesion of TiO₂ to the substrate have been proposed. However, such binders can drastically reduce photocatalytic efficiency.

Finally, the impregnation of a surface with a mixture comprising a photocatalyst can strongly modify the fluid dynamics at the impregnated surface and drastically reduce the photocatalytic efficiency and increase the pressure drop in the air purifier.

Recently, it has been proposed to use composite fibers including polymers and small amounts of photocatalyst to form filters for air purifiers. However, with these techniques, the photocatalyst is trapped in the photocatalytic fiber in a random manner, which considerably reduces its efficiency. Thus a fiber can comprise TiO₂ aggregates or, conversely, areas without TiO₂. In addition, the presence of aggregates in the polymer can hinder subsequent manufacturing steps such as spinning when the substrate is in the form of fibers.

Furthermore, currently most substrates for fixing TiO₂ comprise fibers from glass, ceramic, clay mineral, zeolite, metal plate, cellulose or else activated carbon fibers. However, these substrates are on the one hand particularly fragile and on the other hand very inflexible, which leads to a limitation in the geometry of the purifiers which all look the same at present. Indeed, during spinning, the fibers are drawn, heated and cooled several times. The fibers can then suffer damage such as cracks or even ruptures. In particular, these fibers can be over-drawn and undergo a loss of mass, a pressure drop or even a drop in force. This greatly limits the shaping and geometry of textiles and therefore photocatalysts.

Moreover, during industrialization, it becomes very difficult to ensure good capacity and efficiency of a photocatalyst based on fibers containing TiO₂. On the one hand, in order to ensure good photocatalytic performance, it is necessary to integrate a sufficient amount of photocatalyst into the fiber while ensuring sufficient fluidity of the matrix during spinning and a homogeneous distribution of TiO₂, and on the other hand, it is necessary to ensure optimal spinning so that the fibers preserve their mechanical characteristics and their handling while limiting the pressure drop.

In order to overcome the problems of aggregates and to ensure optimal dispersion of TiO₂, for example, a linear titanium oxide polymer has been proposed in document US2020282387. In this document, the coating is formed by sintering a solution comprising the polymer which can be deposited on a substrate in the form of fibers. However, in this document, the substrate is preferably rough with protruding external surfaces. This allows to improve the adhesion of the TiO₂ to the substrate but accentuates the formation of TiO₂ aggregates on the surface. Furthermore, sintering imparts high rigidity to the structure, which reduces workability and prevents any shaping operation. In addition, such a product is very friable and very fragile which limits in particular the reduction of its thickness to reduce the pressure drop.

Other techniques have been developed, such as for example a method for manufacturing macroscopic TiO₂ fibers by continuous extrusion in unidirectional flow described in document EP3126550. Such a method allows to produce macroscopic TiO₂ fibers on a large scale. However, in order to obtain good photocatalytic performance, the polymer is entirely calcined after the manufacture of the fibers which then contain only TiO₂ and are extremely brittle. Their mechanical resistance after calcination does not allow the application of an air flow powerful enough for its use in an air purifier.

The purpose of the invention is to overcome the disadvantages of the prior art. In particular, the purpose of the invention is to propose a method for manufacturing a multi-component fiber with a high surface concentration of photocatalyst, having a homogeneous distribution of the photocatalyst so as to optimize the efficiency of a photocatalytic air purifier using this fiber, while allowing to offer a malleable fabric that can support different shaping operations while maintaining high air permeability.

### SUMMARY OF THE INVENTION

The invention aims at overcoming these disadvantages.

The invention relates in particular to a method for manufacturing a photocatalytic multi-component fiber including the following steps:
Providing a support mixture, said support mixture including at least one thermoplastic polymer or a thermoplastic polymer precursor;
Providing an active mixture, said active mixture including:
   at least one organic polymer or one organic polymer precursor,
   at least one photocatalyst at a concentration of at least 10% by weight relative to the weight of active mixture,
   at least one coupling agent resistant to oxidation, preferably a silane; or a coupling agent precursor resistant to oxidation,
Spinning a multi-component fiber from support and active mixtures;
Eliminating the at least one organic polymer on the surface of the multi-component fiber so as to generate a photocatalytic multi-component fiber.

The applicant has developed a method capable of generating a multi-component photocatalytic fiber having a high photocatalyst content, a homogeneous surface distribution of the photocatalyst while being deformable and having mechanical characteristics compatible with their use in an air purifier. Indeed, the elimination step allows to form an inorganic surface, preferably predominantly inorganic including the photocatalyst in contact with a thermoplastic polymer support.

The applicant has developed in particular a support mixture and an active mixture which combined allow to ensure the production of a flexible fiber having a high content of the photocatalyst on the surface and a homogeneous distribution on the surface of the photocatalyst so that the photocatalyst aggregates are reduced or non-existent, and that the surface of the fiber is mainly covered with photocatalyst.

Furthermore, such a method also gives the possibility of spinning fibers without breakage, cracking, or else loss of force.

Thus, a method according to the invention allows to meet the needs and in particular to propose the generation of a multi-component photocatalytic fiber with a high surface concentration of photocatalyst so as to optimize the capacity and efficiency of the photocatalyst, and its homogeneous distribution providing a porous multi-component fiber surface with little or no aggregate while being malleable to respond to different shaping operations.

According to other optional characteristics of the method, the latter may optionally include one or more of the following characteristics, alone or in combination:
the elimination of the at least one organic polymer on the surface of the multi-component fiber comprises a surface calcination so as to generate an inorganic surface in contact with a thermoplastic polymer support. This allows on the one hand to activate the photocatalyst and on the other hand to ensure a homogeneous distribution, while providing a porous surface to the photocatalytic multi-component fiber to improve exchanges with the air;
the thermoplastic polymer(s) of the support mixture are selected from thermoplastic polymers having a melting temperature comprised between 100° C. and 350° C. These thermoplastic polymers of the support mixture are preferably biosourced and/or biodegradable;
it includes an extrusion and/or a coextrusion of the support mixture and/or the active mixture;
the support mixture and the active mixture have hot flow indices measured according to standard ISO 1133, such that the difference between the melt flow index of the active mixture and the melt flow index of the support mixture is lower or equal to 20%. This allows better control of the viscosity of the support and active mixtures in order to ensure optimal spinning without breakage and/or loss of force. Furthermore, this can ensure optimized mechanical properties to the photocatalytic multi-component fiber,
during spinning, the multi-component fiber has a sheathing rate comprised between 5% and 50% calculated according to the formula 4e*(D−e)/D². Which can ensure homogeneous distribution of the photocatalyst and reduces the pearl necklace effect.

According to a second object, the invention relates to a photocatalytic multi-component fiber including:
a thermoplastic polymer support, and
An inorganic surface;
the thermoplastic polymeric support including at least one thermoplastic polymer, and the inorganic surface including a coupling agent network, which is resistant to oxidation, associated with a photocatalyst.

According to other optional characteristics of the photocatalytic multi-component fiber, the latter may optionally include one or more of the following characteristics, alone or in combination:
it has a diameter less than or equal to 150 μm,
the inorganic surface has a thickness of at least 500 nm,

According to a third object, the invention relates to a textile including at least one photocatalytic multi-component fiber according to the invention. Such a textile according to the invention is flexible and malleable.

According to a fourth object, the invention relates to a filter including at least one textile according to the invention. Such a filter can have variable geometries, for example pleated, unlike existing filters. Furthermore, a filter according to the invention can be of adapted conformation, for example tailor-made, unlike existing filters.

According to a fifth object, the invention relates to a photocatalytic air purifier equipped with at least one filter according to the invention, a ventilation system and an ultraviolet illumination system capable of illuminating the at least one filter; said ventilator being arranged to convey air from an inlet of the purification system to an outlet of the purification system through the filter.

### BRIEF DESCRIPTION OF THE DRAWINGS

Other characteristics and advantages of the invention will be better understood upon reading the description which follows and with reference to the appended drawings, given by way of illustration and in a non-limiting manner.

FIG. 1 shows a diagram of a method for manufacturing a photocatalytic multi-component fiber according to one embodiment of the invention.

FIG. 2 shows a diagram showing different sections of photocatalytic multi-component fibers according to the invention.

FIG. 3 shows a shot of a photocatalytic multi-component fiber according to one embodiment of the invention, at ×80 magnification by scanning electron microscopy (SEM).

The figures do not necessarily respect scales, particularly in thickness, for illustration purposes.

Aspects of the present invention are described with reference to flow charts and/or block diagrams of methods and apparatuses (systems) according to embodiments of the invention.

In the figures, flow charts and block diagrams illustrate the architecture, functionality and operation of possible implementations of systems and methods according to various embodiments of the present invention. In some implementations, the functions associated with the blocks may appear in a different order than shown in the figures. For example, two blocks shown in succession may, in fact, be executed substantially simultaneously, or the blocks may sometimes be executed in reverse order, depending on the functionality involved.

### DESCRIPTION OF EMBODIMENTS

Below, a summary of the invention and the associated vocabulary are described before presenting the disadvantages of the prior art, then finally showing in more detail how the invention overcomes them.

In the remainder of the description, the expression “multi-component fiber” can correspond to a two-component, three-component fiber or more.

The term “support mixture” within the meaning of the invention may correspond to a mixture intended to form a network in support of the active mixture so that the active mixture rests on, around or in the support mixture. For example, in a core-sheath fiber configuration, a support mixture can correspond to a fiber core.

The term “active mixture” within the meaning of the invention may correspond to a mixture intended to form a preferably inorganic network on the surface of the support mixture and intended to interact with a physical stimulus during the photocatalysis reaction. For example, in a core-sheath fiber configuration, the active mixture may correspond to the fiber sheath.

The term “core” within the meaning of the invention can correspond to an internal part of a fiber such as a core.

The term “sheath” within the meaning of the invention can correspond to an external part of a fiber such as an envelope.

The expression “thermoplastic polymer” within the meaning of the invention may correspond to a polymer which, repeatedly, can be softened or melted under the action of heat and which adopts new shapes by application of heat and pressure.

The expression “thermoplastic polymer precursor” within the meaning of the invention may correspond to a component allowing to start or initiate a polymerization reaction of one or more monomer(s).

The expression “organic polymer” within the meaning of the invention may correspond to a linear, branched or cyclic polymer, the polymer core of which comprises at least one carbon atom.

The term “polymerization” within the meaning of the invention can correspond to the process allowing to convert a monomer or a mixture of monomers into a polymer.

The term “predominantly” within the meaning of the invention can correspond to at least 50%, preferably more than 50%. For example, a predominantly inorganic surface within the meaning of the invention may correspond to a surface comprising, by mass, more inorganic polymer than organic polymer or by mole, more inorganic molecule than organic molecule. A predominantly inorganic surface can for example comprise at least 50% by weight of photocatalyst and coupling agent.

The expression “resistant to oxidation” within the meaning of the invention may correspond to a reduction or limitation of interactions with oxygen so that redox reactions are reduced particularly during photocatalysis.

The expression “substantially equal” within the meaning of the invention can correspond to a value varying by less than 50% relative to the compared value, preferably by less than 40%, even more preferably by less than 30%. When substantially equal is used to compare values then the compared value varies by less than 50% compared to the value taken as reference, preferably by less than 40%, even more preferably by less than 30%.

The invention proposes to take into consideration the existing difficulties associated with unwieldy photocatalytic filters and in particular which can lead to a pressure drop and/or a reduction in the efficiency of the photocatalysts.

In particular, the invention proposes a method for manufacturing a multi-component fiber comprising a polymeric support combined with an inorganic surface, preferably predominantly inorganic. Such a method allows to form a fiber with high mechanical strength capable of being shaped without damaging it then to form a filter having a photocatalytic surface combining high permeability compared to an impregnated textile having the same characteristics (thickness, density, size of fibers) and high photocatalyst content.

Thus, the invention relates to a method for manufacturing a multi-component fiber.

FIG. 1 illustrates an example of a method 100 for manufacturing a photocatalytic multi-component fiber. Such a manufacturing method 100 comprises a step 110 of supplying a support mixture, a step 120 of supplying an active mixture, a spinning step 130, and a step 160 of eliminating at least one organic polymer on the surface of the multi-component fiber. Furthermore, a manufacturing method 100 according to the invention may include a step of forming a fabric 140 and a step 150 of shaping the fabric.

In the example of FIG. 1, the method 100 for manufacturing a photocatalytic multi-component fiber comprises a step 110 of supplying a support mixture. The support mixture includes at least one thermoplastic polymer. Alternatively, the support mixture includes at least one thermoplastic polymer precursor. According to another alternative, the support mixture includes at least one thermoplastic polymer and at least one thermoplastic polymer precursor.

The thermoplastic polymer(s) of the support mixture may be selected from thermoplastic polymers having a melting temperature below 350° C., for example a melting temperature comprised between 100° C. and 350° C. Advantageously, the thermoplastic polymer(s) of the support mixture can be selected from: polypropylene, polyester, polyethylene, polylactic acid, polyamide, polyvinyl, polyacrylate, polybutylene terephthalate, polyhydroxyalkanoates, poly (butylene adipate-co-terephthalate and a mixture thereof. For example, the thermoplastic polymer(s) of the support mixture can be selected from: polyethylene terephthalate, bio-polyethylene terephthalate, bio-polyethylene, biodegradable polyester, and polyhydroxyalkanoate.

Preferably, the thermoplastic polymer(s) of the support mixture can be selected from biosourced and/or biodegradable thermoplastic polymers.

A thermoplastic polymer precursor according to the invention for the support mixture can for example be selected from all thermoplastic polymer precursors having a melting temperature comprised between 100° C. and 350° C. Advantageously, the thermoplastic polymer precursor(s) can be selected from lactides, acrylates, methacrylates, styrenes, and/or lactones.

Advantageously, the thermoplastic polymer(s) of the support mixture and/or the thermoplastic polymer(s) formed from the thermoplastic polymer precursor of the support mixture have good properties for spinning.

A step of providing a support mixture may for example comprise the preparation of a support mixture. In this embodiment, when the support mixture comprises a precursor such as at least one thermoplastic polymer monomer, the step of preparing the support mixture may comprise a polymerization step. A polymerization step can be carried out using a stimulus such as a plasma, an ion bombardment, an electrochemical process, a chemical species (nucleophile, electrophile, etc.), light radiation. Moreover, a polymerization step can be carried out for a predetermined duration and temperature depending on the at least one monomer.

The step of preparing a support mixture can be carried out at a temperature less than or equal to 400° C. and greater than or equal to 100° C.

Optionally, the step of preparing the support mixture may comprise the addition of an additive, for example to improve the resistance of the flexible support. For example a photocatalytic multi-component fiber core in a core-sheath fiber configuration mode.

The method 100 for manufacturing a photocatalytic multi-component fiber comprises a step 120 of supplying an active mixture.

The active mixture includes at least one organic polymer, such as for example at least one thermoplastic polymer. Alternatively, the active mixture includes at least one organic polymer precursor. According to another alternative, the active mixture includes at least one organic polymer and at least one organic polymer precursor.

The polymer(s) of the active mixture may be selected from polymers having a melting temperature comprised between 100° C. and 350° C. Advantageously, the polymer(s) of the active mixture may be selected from: polypropylene, polyester, polyethylene, polylactic acid, polyamide, polyvinyl, polyacrylate, polybutylene terephthalate, polyhydroxyalkanoates, poly (butylene adipate-co-terephthalate and a mixture thereof. For example, the polymer(s) of the active mixture may be selected from: polyethylene terephthalate, bio-polyethylene terephthalate, bio-polyethylene, biodegradable polyester, and polyhydroxyalkanoate.

Preferably, the polymer(s) of the active mixture can be selected from biosourced and/or biodegradable thermoplastic polymers.

Moreover, the polymer(s) of the active mixture may comprise an additional grafted chemical group.

A polymer precursor of the active mixture according to the invention can for example be selected from all polymer precursors having a melting temperature comprised between 100° C. and 350° C. Advantageously, the polymer precursor(s) can be selected from lactides, acrylates, methacrylates, styrenes, and lactones.

In a particular embodiment of the invention, the provision of the active mixture may comprise a step of preparing an active mixture.

A step of preparing an active mixture can be carried out at a temperature less than or equal to 400° C. and greater than or equal to 100° C.

In the case where the preparation of the active mixture comprises at least one thermoplastic polymer monomer, the step of preparing the active mixture may comprise a polymerization step. A polymerization step can be carried out using a stimulus such as a plasma, an ion bombardment, an electrochemical process, a chemical species (nucleophile, electrophile, etc.), light radiation. Moreover, a polymerization step can be carried out for a predetermined duration and temperature depending on the at least one monomer.

In a particular embodiment of the invention, the active mixture includes the same polymers or polymer precursor as the support mixture.

Advantageously, the polymer(s), preferably thermoplastic, of the active mixture and/or the polymer(s) formed from the polymer precursor, preferably thermoplastic, of the active mixture have good properties for spinning.

The active mixture comprises at least one photocatalyst. Preferably, the active mixture comprises at least one photocatalyst at a concentration of at least 10% by weight relative to the weight of active mixture, preferably at least 15%, at least 20%, at least 25%, at least 30%, at least 35%, at least 40%. Preferably, the active mixture comprises a photocatalyst at a concentration less than or equal to 50% by weight relative to the weight of the active mixture, preferably at a concentration less than or equal to 45%. For example, the photocatalyst(s) will be present in the active mixture at a concentration of 10% to 50% by weight relative to the weight of active mixture, preferably at a concentration of 15% to 45% by weight relative to the weight of active mixture, more preferably from 20% to 45% by weight relative to the weight of active mixture and even more preferably at a concentration of 25% to 40%. The photocatalyst can be dosed by gravimetric dispenser.

A photocatalyst within the meaning of the invention can be selected from: transition metals, poor metals, metalloids and their oxide, preferably having photocatalysis properties. For example, a photocatalyst can be selected from AgBr, AgCl, Ag₃PO₄, Ag₂S, AgI, Bi₂O₃, Bi₂S₃, C₃N₄, CdS, CdSe, CdO, Ce₂O₃, CezS₃CoO, CuO, Cu₂O, Cu₂S, CuInS₂, FeTiO₃, Fe₂O₃, GaAs, GaP, In₂S₃, MoS₂Nn₂O₃, NiO, PbO, PdO, RuO₂, SnO₂, SnS, TiO₂, V₂O₅, WS₂WO₃, ZnO, ZnS, ZrS₂ ZnSe, ZrO₂ and a mixture thereof.

The photocatalyst can optionally be doped and/or grafted. For example, a photocatalyst can be pretreated with a hydrophobic treatment.

Preferably, the photocatalyst is TiO₂.

The photocatalyst can be in crystalline form. In the particular case of TiO₂, said photocatalyst can be in the form of anatase or an anatase and rutile mixture or an anatase, rutile and brookite mixture.

The photocatalyst may be in the form of nanoparticles having an average diameter of 2 nm to 100 nm, preferably 5 nm to 75 nm, more preferably between 10 nm and 50 nm. The photocatalyst can be in powder form or in the form of a precursor in solution.

According to one embodiment, the photocatalyst is incorporated into the active mixture when it is in the molten state. This limits and minimizes the formation of aggregate.

The active mixture comprises at least one coupling agent or a coupling agent precursor. Preferably, a coupling agent according to the invention resistant to oxidation.

A coupling agent may comprise a chemical function allowing to produce a chemical bond with at least one photocatalyst, preferably with TiO₂. A coupling agent may comprise a chemical function allowing a polymerization reaction of the coupling agent so as to form a network. A coupling agent may comprise a chemical function capable of producing a chemical bond with a thermoplastic polymer of the support mixture.

A coupling agent may be selected from one or more geopolymers or one or more geopolymer precursors.

A coupling agent can be selected from silanes, or siloxanes for example.

Preferably the coupling agent is selected from: vinyltrimethoxysilane, polydimethylsiloxane, tetraethoxysilane, tetramethoxysilane, tetrapropoxysilane n-propyltriethoxysilane, ethyltrimethoxysilane, methyltriethoxysilane, propyltrimethoxysilane, propyltriethoxysilane, phenyltrimethoxysilane, phenyltriethoxysilane, trimethoxyvinylsilane, triethoxyvinylsilane, vinyltriethoxysilane, vinyltris (β-methoxyethoxy) silane, β-(3,4-epoxycyclohexyl)ethyltrimethoxysilane, γ-(2-aziridine) aminopropyltrimethoxysilane, γ-glycidoxypropyltrimethoxysilane, γ-glycidoxypropyltrimethyldiethoxysilane, γ-glycidoxypropyltriethoxysilane, γ-methylpropenylpropyldimethoxysilane, γ-methylpropenyltrimethoxysilane, γ-methylpropenylpropyldiethoxysilane, γ-methylpropenylpropyltriethoxysilane, N-β (aziridine)γ-aminopropylmethyldimethoxysilane, N-β(aziridine)γ-aminopropyltrimethoxysilane, N-β(aziridine)γ-aminopropyltriethoxysilane, N-β-3-acryloxypropyltrimethoxysilane, 3-(aminoethyl)-γ-aminopropyltrimethoxysilane aminopropyltrimethoxysilane, 3-aminopropyltriethoxysilane, N-phenyl-γ-aminopropyltrimethoxysilane, γ-chloropropyltrimethoxysilane, γ-hydrothiopropyltrimethoxysilane, bis[3-(triethoxysilyl) propyl] tetrasulfide (TESPT) and bis[3-(triethoxysilyl)propyl]-disulfide, and preferably is 3-acryloxypropyltrimethoxysilane, 3-chloropropyltrimethoxysilane, 3-mercaptopropyltrimethoxysilane, 3-methacryloxypropyltrimethoxysilane, 3-acryloxypropyltrimethoxysilane and a combination thereof. These coupling agents allow good dispersion of charges and to simplify the compounding process. Preferably, a coupling agent allows to form a network which is inorganic, which allows to resist photocatalysis. Preferably the inorganic network is crosslinked. It preferably includes polymer chains bound together by bonds of lower molecular mass. It can then form a three-dimensional network. Advantageously, an inorganic network allows to resist oxidation and therefore photocatalysis.

A coupling agent precursor can be selected from: silica, siloxane, silanol.

The active mixture comprises at least one coupling agent at a concentration of at least 0.75% by weight relative to the weight of active mixture, preferably at least 1.5%, at least 2%, at least 2.5% at least 3%. Preferably the active mixture comprises a coupling agent at a concentration less than or equal to 10% by weight relative to the weight of the active mixture, preferably at a concentration less than or equal to 8%. For example, the active mixture comprises at least one coupling agent at a concentration of 0.75% to 10% by weight relative to the weight of active mixture, preferably at least 1.5% and 8% by weight relative to the weight of active mixture.

Advantageously, the addition of a coupling agent to the active mixture allows to reduce the viscosity of the active mixture relative to the viscosity of the same active mixture without coupling agent, measured by rheometer according to standard ISO 1628.

The active mixture may include a mass ratio of the at least one coupling agent to the at least one photocatalyst comprised between 1/50 and 5/1. Preferably, the active mixture may include a mass ratio of the at least one coupling agent to the at least one photocatalyst comprised between 1/20 and 2/1. More preferably, the active mixture may include a mass ratio of the at least one coupling agent to the at least one photocatalyst comprised between 1/10 and 1.

According to one embodiment, the coupling agent is incorporated into the active mixture by means of a pump, for example a peristaltic pump, by injection.

Preferably, the photocatalyst used in the context of the invention is previously combined with the coupling agent. Thus, according to a preferred embodiment of the invention, the coupling agent is added to the active mixture when it is coupled to the photocatalyst. Thus, in this case, the method may include a step of coupling the photocatalyst to the coupling agent or a step of providing a photocatalyst coupled to the coupling agent.

The step of supplying a support mixture and/or the step of supplying an active mixture can be carried out by a mixer (compounder), a kneader, an extruder and/or a dispenser of the gravimetric metering type and/or volumetric dispenser.

In a preferred embodiment, the step of providing a support mixture and/or the step of providing an active mixture comprises an extrusion and/or coextrusion. The step of providing a support mixture and/or the step of providing an active mixture may comprise multiple extrusion. Multiple can be greater than or equal to two. Multiple extrusion allows to improve the dispersion of the photocatalyst in the active mixture as well as the incorporation of the coupling agent.

When the step of providing a support mixture comprises an extrusion of the support mixture, the extrusion step is configured so that the support mixture has a melt flow index (MFI) substantially equal to the melt flow index of the active mixture, measured according to standard ISO 1133 (as measured at 230° C. for a standard mass of 2160 g). For example, the support mixture has a melt flow index (MFI) whose difference with the melt flow index of the active mixture is less than or equal to 20%, preferably less than or equal to 10% and even more preferably less than or equal to 5%. Thus the difference between the MFI of the support mixture and the MFI of the active mixture is small and preferably less than or equal to 15%.

The melt flow index of the active mixture can for example be comprised between 5 g/10 min and 1500 g/10 min measured according to standard ISO 1133. For example, the melt flow index of the support mixture can be comprised between 9 g/10 min and 15 g/10 min. In one embodiment, the MFI of the support mixture may be 12 g/10 min and the MFI of the active mixture may be 27 g/10 min. In another embodiment, the MFI of the support mixture may be 12 g/10 min and the MFI of the active mixture may be 5 g/10 min. In another embodiment, the MFI of the support mixture may be 12 g/10 min and the MFI of the active mixture may be 11 g/10 min.

The step of preparing an active mixture can be carried out at a temperature less than or equal to 400° C. and greater than or equal to 100° C., preferably less than or equal to 300° C., more preferably less than or equal to 250° C.

A method 100 for manufacturing a photocatalytic multi-component fiber may include a spinning step 130. The spinning step is preferably carried out from a support mixture and an active mixture.

A spinning step can be carried out under hear by melt spinning (for quenching on a wheel), spun bond (for cold drawing) and/or meltblown (for hot drawing).

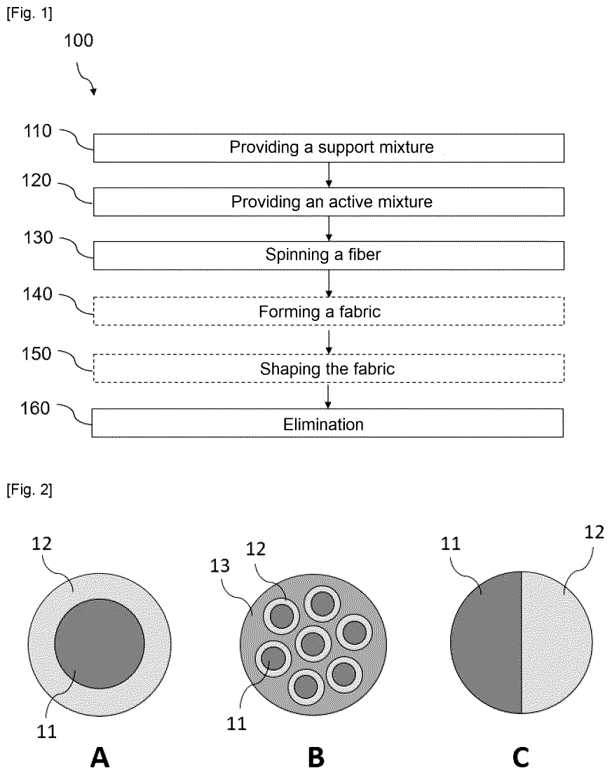

FIG. 2 shows examples of spinning configurations according to the present invention. This figure shows some of the possible configurations with a core-sheath configuration in FIG. 2A; an island-in-the-sea type configuration in FIG. 2B; a side-by-side type configuration in FIG. 2C.

The configurations illustrated in FIGS. 2A and 2B allow to form one or more fibers including a core-type thermoplastic polymer support 11 and an inorganic surface 12, preferably predominantly inorganic, of the inorganic sheath type. As illustrated in FIG. 2B, the multi-component fiber can include three phases: a thermoplastic polymer support 11, an inorganic, preferably predominantly inorganic surface 12 and a sacrificial matrix 13. Indeed, in a particular embodiment, the multi-component fiber may include a sacrificial matrix generally removed before the step of eliminating at least one organic polymer.

As illustrated in FIG. 2C, the multi-component fiber may include a support 11 in contact with an inorganic, preferably predominantly inorganic surface 12.

An additional wire drawing step can be carried out under cold and/or hot conditions in order to reduce the diameter of the wire obtained.

Preferably the spinning step can be carried out in order to achieve a fiber diameter comprised between 2 μm and 150 μm.

In order to avoid damage such as cracks, ruptures or else a loss of mass or else a drop in force, the spinning step is carried out so that the fiber has a sheathing rate comprised between 5 and 50%, preferably between 7 and 30% measured according to the formula 4e*(D−e)/D² by microscopy (SEM). With a sheathing percentage equal to the surface area of the sheathing (Sg) divided by the total surface area (St), with Sg=St−Score, that is to say Sg=π*D²/4−π*(D−2e)²/4 either Sg=π*(4De−4e²)/4, or Sg=π*e*(De) and therefore the sheathing percentage corresponds to 4e*(D−e)/D². With D the external diameter of the sheathing and e the thickness of the sheathing. Thanks to a controlled sheathing rate, the pearl necklace effect (inhomogeneous distribution of the photocatalyst) is minimized.

Advantageously, the spinning step can be carried out by melting and can comprise hot drawing which can be carried out by means of at least one extruder comprising one or more heating zones. Preferably, the melt spinning step comprises an extruder for the support mixture and an extruder for the active mixture.

The spinning step can be configured to vary the ratio (for example mass) of support mixture relative to the active mixture. This can allow to reduce the diameter of the fiber and, for example, to vary the core ratio relative to the sheath ratio for a core-sheath type fiber. Furthermore, the fiber obtained thanks to the preparation steps of the support mixture and the active mixture has a homogeneous texture (measured by SEM microscopy) during the spinning step which demonstrates good dispersion of charges and a homogeneous distribution of the photocatalyst on the surface. The fibers can be air-cooled and directly wound.

The spinning step may comprise a drawing step. Drawing can be carried out, preferably at a temperature comprised between the glass transition temperature and the melting temperature of the polymers of the support and/or active mixtures. In this embodiment, the coil is unwound and passed through a first drawing bench at a speed V1, the fiber is reheated, preferably between 80° C. and 140° C., and drawn again using a second draw bench at a speed V2 before being wound again. The drawing ratio V2/V1 allows the diameter to be varied. Drawing allows to refine the diameter of the fiber and to improve its mechanical properties.

The method 100 for manufacturing a multi-component fiber may include a step of forming a fabric 140 from the multi-component fiber. The step of forming the fabric 140 from the multi-component fiber may or may not include a weaving or knitting step.

The step of forming the fabric 140 can be carried out from a single multi-component fiber, several multi-component fibers or else a sheet of multi-component fibers. The fabric formed may correspond to a non-woven fabric or a fabric woven in a flat, tubular shape and in some other three-dimensional patterns. As with other textile structures, various properties can be incorporated into the mesh to meet design objectives which may include increased flexibility, increased strength, reduced thickness, improved handling and improved mechanical strength.

The method 100 for manufacturing a multi-component fiber may include a step 150 of shaping the fabric. This shaping step 150 allows to form a fabric which can have a great diversity of conformation thanks to the flexibility of the multi-component fiber including thermoplastic polymers.

The method 100 for manufacturing a multi-component fiber comprises a step 160 of eliminating at least one polymer on the surface of the multi-component fiber. Preferably organic polymer. The elimination step allows to generate a photocatalytic multi-component fiber. Advantageously, the elimination step includes a surface treatment of the multi-component fiber.

Preferably, the step of eliminating the at least one polymer, which is preferably organic, on the surface of the multi-component fiber allows to generate an inorganic, preferably predominantly inorganic surface in contact with a thermoplastic polymer support. This allows, for example, to form an inorganic sheath surrounding a thermoplastic polymer core in a core-sheath fiber configuration. The inorganic, preferably predominantly inorganic surface generated may comprise the presence of organic polymer. However, the remaining organic polymer will be a minority by weight in the inorganic surface. Indeed, the inorganic surface preferably includes at least 50% by weight of photocatalyst and coupling agent.

A step of eliminating the at least one polymer, which is preferably organic, on the surface may comprise a heat treatment, a chemical treatment, a plasma, that is preferably localized, that is to say on the surface of the multi-fiber component. Preferably, the treatment allows to eliminate at least part of the organic polymer from the active mixture over a depth of at least 500 nm, more preferably at least 1 μm, even more preferably at least 2 μm. Preferably, the treatment allows to eliminate at least part of the organic polymer from the active mixture over a depth of at most 50 μm, more preferably at most 30 μm, even more preferably at most 20 μm. For example, the elimination step allows to degrade at least part of the at least one organic polymer of the active mixture over a depth of 500 nm to 50 μm, preferably 1 μm to 30 μm, and so more preferably from 2 μm to 20 μm.

In the particular embodiment illustrated in connection with FIG. 2B, the sacrificial matrix can be eliminated prior to the elimination step 160 also according to a chemical, thermal, or else plasma treatment.

Moreover, the step of eliminating at least one polymer, which is preferably organic, from the surface of the multi-component fiber, can be partial or total. Partial elimination may correspond to limited elimination over the depth of the multi-component fiber of the organic polymer(s).

A heat treatment can be selected from: UV (ultraviolet) or IR (infrared) radiation treatment, convection heating, conduction heating. Preferably, heat treatment comprises calcination. A heat treatment is preferably localized on the surface and more preferably over a depth of at least 500 nm. Heat treatment can be carried out at a temperature comprised between 350° C. and 550° C. A heat treatment can be applied for a duration comprised between 0.5 hours and 7 hours.

A chemical treatment can be selected from a treatment using a reactive species which is in solid, liquid, or gas form and which will allow to eliminate the polymer from the active mixture, for example, a treatment in an oxidizing liquid of the type oxygenated water.

A plasma treatment will generally include gas ionization. Plasma treatment may for example include an application of electric or magnetic fields through a gas so as to form a plasma capable of oxidizing the surface of the multi-component fiber.

Elimination of the preferably organic polymer on the surface allows to increase the level of photocatalyst on the surface, which allows to increase the efficiency of photocatalysis and therefore to greatly improve the photocatalytic properties of the multi-component fiber.

According to another aspect, the invention relates to a photocatalytic multi-component fiber.

A multi-component photocatalytic fiber according to the invention can be obtained by the method according to the invention. Preferably, it is directly obtained by the method according to the invention.

A multi-component fiber comprises a support comprising at least one thermoplastic polymer and an inorganic surface, preferably a predominantly inorganic surface.

In a particular embodiment, a photocatalytic multi-component fiber according to the invention comprises a thermoplastic polymeric core and an inorganic sheath.

In an optional embodiment, the photocatalytic multi-component fiber may comprise, preferably between the core and the sheath, at least one intermediate sheath. An intermediate sheath may comprise another thermoplastic polymer, inorganic materials (for example silica), and/or coupling agents. An intermediate sheath can, for example, help protect the core and increase the lifespan of the multi-component fiber. The multi-component fiber may also comprise a sacrificial matrix.

The thermoplastic polymer core of the multi-component fiber includes thermoplastic polymers. The polymeric core of the multi-component fiber may comprise additives. The thickness of the core of the multi-component fiber can be comprised between 1 μm and 150 μm.

The sheath of the multi-component fiber includes a network of oxidation-resistant coupling agent and photocatalyst. Preferably a network of silica and titanium. The surface thickness of the photocatalyst is preferably comprised between 300 nm and 20 μm. Advantageously, the sheath of the multi-component fiber is at least 50% inorganic, preferably at least 60%, more preferably at least 70% and even more preferably at least 80%. The sheath of the multi-component fiber may be less than 100% inorganic, for example preferably less than 95%. Advantageously, the inorganic sheath has at least 5% by weight of photocatalyst, preferably at least 10% by weight of photocatalyst, preferably at least 15% by weight of photocatalyst, more preferably at least 20% by weight of photocatalyst and even more preferably at least 25%. For example, the inorganic sheath has less than 95% by weight of photocatalyst, preferably less than 90% by weight of photocatalyst and more preferably less than 85% by weight of photocatalyst, preferably after removal of the organic polymer from the active mixture. The thickness of the sheath of the multi-component fiber can be comprised between 300 nm and 20 μm. The photocatalytic multi-component fiber according to the invention has a thickness, preferably a diameter, less than or equal to 150 μm, preferably less than or equal to 100 μm, more preferably less than or equal to 50 μm, even more preferably less than or equal to 10 μm. For example, the photocatalytic multi-component fiber has a thickness, preferably a diameter, of at least 1 μm.

Preferably, the multi-component fiber has a predominantly inorganic surface thickness, greater than or equal to 500 nm.

The multi-component fiber has a surface. Preferably the surface of the multi-component fiber is homogeneous in composition (MEB/EDX for Energy Dispersive X-ray). Preferably, after treatment, the multi-component fiber is porous with a specific surface area of at least 10 m²/g (measured by BET analysis). The multi-component fiber can have a specific surface area of at most 500 m²/g. Preferably the multi-component fiber can have a specific surface area comprised between 10 m²/g and 500 m²/g.

According to another aspect, the invention relates to a textile including at least one photocatalytic multi-component fiber according to the invention.

A textile according to the invention can correspond to a textile usable in all fields such as for example the clothing, decorative, or industrial fields. It can be used in water and/or air treatment methods, in particular via photocatalysis for the purification of water and/or air, or for its anti-bacterial and self-cleaning properties. Such a textile could be involved in the destruction of preferably volatile organic compounds.

Advantageously, a textile according to the invention is capable of being deformed in order to have different geometries. Thus a textile according to the invention is flexible in particular before the step of eliminating at least one polymer, preferably organic on the surface. The deformation can be by folding, twisting, twisted without the textile breaking or losing photocatalysis efficiency. Thus, a filter according to the present invention has increased efficiency and photocatalytic capacity.

According to another aspect, the invention relates to a filter including at least one textile according to the invention.

According to another aspect, the invention relates to a photocatalytic fluid purifier comprising at least one filter according to the invention. By fluid it can for example be air or water, preferably air. Preferably the filter is arranged between a fluid inlet and a fluid outlet of the purifier. A purifier may also comprise a ventilation system.

The ventilation system may be arranged to convey fluid from an inlet of the purifier to an outlet of the purifier. A ventilation system is in particular arranged so as to generate a flow of air passing through the filter according to the invention. A ventilation system may for example comprise at least one ventilator equipped with one or more propellers, blades and/or turbines which can be arranged in different positions. Preferably, the ventilation system is arranged so that a flow of air, preferably filtered, leaving the purifier contains a reduced level of VOC, allergen, pollutant and/or pathogen compared to air entering the purifier. Advantageously, the speed of the ventilation system can be adapted. Thus different power levels of the purifier can be defined. This allows, in the event of heavy pollution, for example, to increase the power of the purifier without it operating continuously at maximum power.

A purifier may also comprise an illumination system.

An illumination system preferably comprises an ultraviolet illumination system capable of illuminating the at least one filter according to the invention. Preferably the wavelength of the illumination system is included in the range(s) of U.V.A, U.V.B and/or U.V.C, that is to say from 100 to 400 nm. Advantageously, the illumination system can be centered on one or more bands, for example from 100 nm to 280 nm, from 280 nm to 320 nm, from 320 nm to 400 nm or any other bands allowing to center U.V.A and U.V.B or U.V.B and U.V.C or any other combinations between U.V.A, U.V.B, U.V.C. UV radiation allows the photocatalyst to be activated. The illumination system may for example comprise a U.V lamp.

A purifier may comprise one or more sensors. For example, it can be a pressure sensor, a pollution sensor, a temperature sensor, a particle detection sensor, a concentration sensor, a consumption sensor, and an obstruction sensor.

A purifier may comprise a processing module configured to regulate the speed of the ventilation system and/or the intensity of the lighting system.

A purifier may comprise a communication module configured to communicate between the sensor(s) and the ventilation and/or lighting system through a communication network. The communication module can for example be configured to emit a message containing at least one indication of the measured pressure difference, of the filter obstruction level, an indication of a pollution level, an indication of a depollution level, a type of particle detected, an abnormal rise in temperature within the purifier, and/or abnormal current consumption of the purifier.

### EXAMPLES

### Component

Thermoplastic polymer of the support mixture: polypropylene
Organic polymer of the active mixture: polypropylene
Photocatalyst: TiO₂ 40%, in crystalline form: mixture of anatase and rutile
Coupling agent: polysiloxane

### Compounding

Compounding is carried out using a co-kneader with mixing capacity. It may be a single-screw co-kneader driven by a rotational and translational movement or a twin-screw co-kneader.

The co-mixer may comprise several (gravimetric and/or volumetric) dispensers and one or more heating zones.

The polymers of the active mixture, the coupling agent and possibly additives are introduced into the co-kneader using a hopper, and a dry mixture is produced. The photocatalyst is introduced using the gravimetric dispenser and a transverse feed to ensure incorporation of the photocatalyst in the molten state.

A second gravimetric dispenser will be filled with the active mixture.

The support mixture is also produced using a single or twin-screw extruder.

The mixture is carried out at a temperature comprised between 15° and 300° C.

### Spinning

At the end of the mixture, a spinning step is carried out using a transport belt and bobbin(s). The speeds of each extruder allow to vary the polymer ratio of the support mixture/polymer of the active mixture.

The mechanical characterization of the fibers is carried out according to standard ISO 5079.

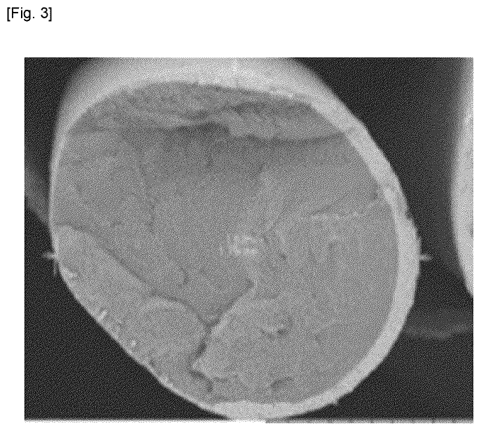

As presented in FIG. 3, the photocatalytic multi-component fibers according to the invention are much more homogeneous in composition, more loaded with photocatalyst, and have few aggregates (MEB). Thus, it is possible to provide more active material even with sheathing rates greater than 25%. Moreover, their diameter decreases with the presence of a drawing step while maintaining good mechanical properties. Indeed, the fibers are more flexible and more resistant while having a diameter of less than 150 μm.

Furthermore, when the manufacturing method includes a prior step of grafting the photocatalyst with the coupling agent, the drawing ratios are optimized (8 versus 6) and the diameters are also reduced.

The photocatalytic multi-component fiber according to the invention has improved mechanical characteristics. The multi-component fiber has optimized flexibility as well as improved malleability.

TABLE 1

| | Multi- | Multi- | Multi- |
|---|---|---|---|
| | component | component | component |
| | fiber-F3 | fiber-F4 | fiber-F16 |
| Emod (MPa) | 4608 | 3923 | 1930 |
| Count (tex) | 20 | 10 | 14 |
| F_{H} (N) | 8.2 | 4.1 | 2.5 |
| Deformability (%) | 113.8 | 29.6 | 26.3 |
| σ_{H} (MPa) | 452.8 | 456.0 | 199.9 |
| Sheathing rate (%) | 13 | 13 | 26 |
| TiO₂ charge (% by weight | 40 | 40 | 40 |
| of active mixture) | | | |
| before calcination | | | |
| Coupling agent (% by | 10 | 10 | 10 |
| weight of active mixture) | | | |

The invention may be the subject of numerous variants and applications other than those described above. In particular, unless otherwise indicated, the different structural and functional characteristics of each of the implementations described above should not be considered as combined and/or closely and/or inextricably related to each other, but on the contrary as simple juxtapositions. Furthermore, the structural and/or functional characteristics of the different embodiments described above may be the subject in whole or in part of any different juxtaposition or of any different combination.

1. A method for manufacturing a photocatalytic multi-component fiber including the following steps:
Providing a support mixture, said support mixture including at least one thermoplastic polymer or a thermoplastic polymer precursor;
Providing an active mixture, said active mixture including:
at least one organic polymer or one organic polymer precursor,
at least one photocatalyst at a concentration of at least 10% by weight relative to the weight of active mixture,
at least one coupling agent resistant to oxidation, preferably a silane; or a coupling agent precursor resistant to oxidation,
Spinning a multi-component fiber from support and active mixtures;
Eliminating the at least one organic polymer on a surface of the multi-component fiber so as to generate a photocatalytic multi-component fiber.

2. The method for manufacturing a photocatalytic multi-component fiber according to claim 1, wherein the elimination of the at least one organic polymer on the surface of the multi-component fiber comprises a surface calcination so as to generate an inorganic surface in contact with a thermoplastic polymer support.

3. The method for manufacturing a photocatalytic multi-component fiber according to claim 1, wherein the thermoplastic polymer(s) of the support mixture are selected from thermoplastic polymers having a melting temperature between 100° C. and 350° C.

4. The method for manufacturing a photocatalytic multi-component fiber according to claim 1, further comprising an extrusion and/or a coextrusion of the support mixture and/or the active mixture.

5. The method for manufacturing a photocatalytic multi-component fiber according to claim 1, wherein during spinning the multi-component fiber has a sheathing rate between 5% and 50% calculated according to the formula 4e*(D−e)/D².

6. A photocatalytic multi-component fiber including:
A support mixture corresponding to a thermoplastic polymer support including at least on thermoplastic polymer, and
An active mixture corresponding to an inorganic surface; the inorganic surface including a network of coupling agent, which is resistant to oxidation, and photocatalyst, the photocatalytic multi-component fiber combining the support mixture and the active mixture spun.

7. The photocatalytic multi-component fiber according to claim 6, wherein it has a diameter less than or equal to 150 μm.

8. The photocatalytic multi-component fiber according to claim 6, wherein it has an inorganic surface with a thickness of at least 500 nm.

9. A textile including at least one photocatalytic multi-component fiber according to claim 6.

10. A filter including at least one textile according to claim 9.

11. A photocatalytic air purification system provided with at least one filter according to claim 10, a ventilation system and an ultraviolet illumination system capable of illuminating the at least one filter; said ventilator being arranged to convey air from an inlet of the purification system to an outlet of the purification system through the filter.
